(19) United States
(12) Patent Application Publication
(10) Pub. No.: US 2023/0301529 A1
(43) Pub. Date: Sep. 28, 2023

(54) CONTINUOUS BLOOD PRESSURE MONITORING

(71) Applicant: BULUEDOP MEDICAL, LTD, Nottinghamshire (GB)

(72) Inventors: Michael Graeme TAYLOR, London (GB); Patrick W. KELLY, Sioux Falls, SD (US)

(21) Appl. No.: 18/021,384

(22) PCT Filed: Aug. 19, 2021
(86) PCT No.: PCT/GB2021/052157

§ 371 Date: Feb. 14, 2023

Related U.S. Application Data
(60) Provisional application No. 63/068,951, filed on Aug. 21, 2020.
(60) Provisional application No. 63/222,944, filed on Jul. 16, 2021.

(51) Int. Cl. A61B 5/022; A61B 5/026; A61B 5/021

(52) U.S. Cl. CPC A61B 5/02225; A61B 5/026; A61B 5/02108; A61B 5/02438

(57) ABSTRACT

This disclosure enables non-invasively obtaining a blood pressure measurement of a person in a repeatable manner in order to provide frequent updates to the blood pressure measurement, while 1) being comfortable for the person when the person is conscious, 2) enabling relaxation of the person, 3) allowing the person to fall asleep, or 4) avoiding waking the person if the person is already asleep. After a first set of blood flow waveform data associated with a blood pressure measurement has been acquired (304), a second set of waveform data is received (306), a parameter change in the second waveform data is identified (308), the parameter change is correlated to the blood pressure measurement (310) so as to generate a virtual blood pressure (312), and an action is taken (314) based on the virtual blood pressure.

### TECHNICAL FIELD

This disclosure relates to monitoring blood pressure.

### BACKGROUND

Medical practice once asked if the patient “had a pulse” when assessing a person's health. But as medicine became more refined, we now ask what is the patient's blood pressure; because, much like their heart rate, a person's blood pressure is one of the earliest changing signs that can tell a health care provider that a person is either doing well or something is going wrong. In critically ill patients, patients under general anesthesia, and patients being sedated, monitoring their blood pressure frequently is critical to good outcomes. That is, blood pressure is a critical (or vital) sign. Thus, we call blood pressure a “vital” sign.

Because frequent blood pressure monitoring is necessary in so many situations, we often place “art lines”, a catheter that penetrates through the skin into usually the radial artery, in order to continuously obtain an accurate blood pressure. Another way we do this, although less accurate, is with a blood pressure cuff placed on the arm or leg. For near continuous monitoring (e.g., every 15 minutes), the cuff goes through an inflation and release cycle to determine blood pressure.

While it can eventually be tolerated, an arterial line in the wrist is unpleasant/uncomfortable (affecting a patient's comfort level, whether awake or asleep). And a blood pressure cuff being inflated every 15 minutes to 200 mmHg is not something one can ignore or sleep through. Furthermore, arterial lines can cause infections, arterial thrombosis, hand ischemia, and many other health issues. And blood pressure cuffs are sources for infection transfer, are uncomfortable, and lead to sleep deprivation and prolonged hospitalization. And while both of these devices/methods can obtain blood pressure, each comes at a significant cost to the patient and society. Therefore, there exists a need for continuous blood pressure monitoring without invasive arterial lines or repeated uncomfortable cycling of a blood pressure cuff.

A blood pressure measurement of a person (e.g., a patient or target) is a measurement of a pressure of a volume of blood of the person against a wall of a blood vessel (e.g., a large artery) of the person. This pressure results from a heart of the person pumping the volume of blood through the blood vessel and the blood vessel compliance therewith. The blood pressure measurement is usually expressed in terms of a systolic pressure measurement (e.g., a maximum pressure measurement obtained during a heartbeat) over a diastolic pressure measurement (e.g., a minimum pressure measurement obtained between two heartbeats) in a cardiac cycle of the person. The blood pressure measurement is measured in millimeters of mercury (mmHg) above a surrounding atmospheric pressure (though an aneroid device or an electronic device does not actually contain mercury).

The blood pressure measurement is one of various vital signs of the person—together with a respiratory rate measurement, a heart rate measurement, an oxygen saturation measurement, and a body temperature measurement that a healthcare professional (e.g., a physician, a nurse) may use in evaluating the person. At rest, the person may have the blood pressure measurement of about 120 millimeters of mercury systolic over about 80 millimeters of mercury diastolic, commonly denoted as “120/80 mmHg.”

The blood pressure measurement may be non-invasively obtained from a sphygmomanometer having an inflatable cuff and a display (e.g., a digital display, an analog display) when the inflatable cuff is worn on the person (e.g., an upper arm). The inflatable cuff is inflated to a pressure above a maximum expected for the person, and then slowly deflated while sensing various pulses in pressure within the inflatable cuff in order to sense the systolic pressure measurement and the diastolic pressure measurement. This approach to non-invasively obtaining the blood pressure measurement is commonly known as an oscillometric method, which can be repeated to provide various frequent updates to the blood pressure measurement. However, this frequent repetition may (1) be uncomfortable (e.g., painful) for the person when the person is conscious, (2) prevent relaxation of the person, (3) deter the person from falling asleep, or (4) awake the person if the person is already asleep.

### SUMMARY

There is a new medical term called “sleep hygiene”, where there is a targeted effort to make sure patient(s) sleep while being cared for (in the hospital or at home). Sleep deprivation has been clearly associated with confusion that leads to added test, and prolonged hospital stays or return visits. And one of the biggest culprits to sleep deprivation is the blood pressure cuff. Broadly, this disclosure enables non-invasively obtaining the blood pressure measurement in a repeatable manner in order to provide frequent updates to the blood pressure measurement, while (1) being comfortable for the person when the person is conscious, (2) enabling relaxation of the person, (3) allowing the person to fall asleep, or (4) avoiding waking the person if the person is already asleep.

At least some of the above-noted technological benefits can be obtained from a sensor (e.g., an ultrasonic sensor, a continuous-wave sensor, an ultrasonic Doppler sensor, an oxygen saturation sensor-pulse oximeter) configured to sense a blood flow that arises from the heart of the person contracting. If the sensor is housed via a wrist band (or another wearable device) and worn on the person (e.g., a limb, a torso, a head, a wrist, a finger, a toe, an upper arm), then the sensor can sense the blood flow and thereby generate a waveform of the blood flow and communicate the waveform to a processor. In response, the processor can process the waveform and thereby predict, forecast, or estimate a likely change in the blood pressure (or heart rate) measurement (the processor generates a “virtual blood pressure” measurement, from the blood flow waveforms and a baseline blood pressure measurement) and hence request a repeat blood pressure (or heart rate) measurement for the person via an inflatable cuff only when an indication of a significant change in the blood pressure (or heart rate) measurement is sensed, where the indication satisfies or does not satisfy a preset or predetermined threshold for the blood pressure (or heart rate) measurement. Therefore, this approach can significantly reduce an amount of blood pressure (or heart rate) measurements via the intrusive oscillometric method required from the person and therefore (1) be comfortable for the person when the person is conscious, (2) enable relaxation of the person, (3) allow the person to fall asleep, or (4) avoid waking the person if the person is already asleep. And this approach provides an advantage in the management of hypertension. By providing a mechanism for continuous monitoring, hypertension patients can have a clearer picture of how their lifestyle and any medications (related or not related to their hypertension) affect their blood pressure and be presented with adjustments as needed, if desired. Furthermore, this can provide their physicians with greater insight into treatment options that would be most effective. These are treatment features not fully available when the patient does not have the ability of continuous blood pressure monitoring.

In an embodiment, a method comprises: instructing, by a processor, a sensor worn on a target to monitor a first blood flow of the target during a time period including a cardiac cycle of the target such that the sensor generates a first set of waveform data representing the cardiac cycle during the time period, where the first set of waveform data is associated with a blood pressure measurement; receiving, by the processor, a second set of waveform data for a second blood flow from the sensor after the first set of waveform data is generated by the sensor; identifying, by the processor, a parameter change in the second set of waveform data relative to the first set of waveform data; correlating, by the processor, the parameter change to the blood pressure measurement such that a virtual blood pressure (a calculated value based on the blood pressure measurement and parameters of the waveform data sets) for the second blood flow is generated; and taking, by the processor, an action based on the virtual blood pressure.

In an embodiment, a device comprises: a processor programmed to: instruct a sensor worn on a target to monitor a first blood flow of the target during a time period including a cardiac cycle of the target such that the sensor generates a first set of waveform data representing the cardiac cycle during the time period, wherein the first set of waveform data is associated with a blood pressure measurement; receive a second set of waveform data for a second blood flow from the sensor after the first set of waveform data is generated by the sensor; identify a parameter change in the second set of waveform data relative to the first set of waveform data; correlate the parameter change to the blood pressure measurement such that a virtual blood pressure (a calculated value based on the blood pressure measurement and parameters of the waveform data sets) for the second blood flow is generated; and take an action based on the virtual blood pressure.

### DESCRIPTION OF DRAWINGS

FIG. 1 illustrates a schematic diagram of an embodiment a processor in communication with a memory, a sensor, and a sphygmomanometer according to various principles of this disclosure.

FIG. 2 illustrates a 3-dimensional diagram of an embodiment of a device configured for measuring a blood pressure of a patient pursuant to FIG. 1 according to various principles of this disclosure.

FIG. 3 illustrates a flowchart of an embodiment of a process for generating a virtual blood pressure (or heart rate) pursuant to FIGS. 1-2 according to various principles of this disclosure.

FIG. 4 illustrates a wave diagram of an embodiment of a blood flow waveform pursuant to FIGS. 1-3 according to various principles of this disclosure.

FIG. 5 shows a block schematic of peripheral vasculature.

FIG. 6 shows an electrical circuit equivalent of the FIG. 5 schematic.

FIG. 7 illustrates systolic acceleration.

FIG. 8 illustrates a series of waveforms with different time compression ratios (compressed, normal, and dilated).

FIG. 9 illustrates the linear relationship of patient specific parameters used in a second embodiment.

FIG. 10 illustrates the monitoring process using the linear relationship of patient specific parameters of the second embodiment.

FIG. 11 shows another diagram of the device for measuring a blood pressure of a patient.

### DETAILED DESCRIPTION

Generally, this disclosure enables non-invasively obtaining the blood pressure measurement in a repeatable manner in order to provide frequent updates to the blood pressure measurement, while (1) being comfortable for the person when the person is conscious, (2) enabling relaxation of the person, (3) allowing the person to fall asleep, or (4) avoiding waking the person if the person is already asleep. For example, at least some of these technological benefits can be obtained from a sensor (e.g., an ultrasonic sensor, a continuous-wave sensor, an ultrasonic Doppler sensor, an oxygen saturation sensor-pulse oximeter, photometric sensors (to obtain, for example, a photo (or digital) plesmographgram) on such things but not limited to (smartphones, smartwatches, tablets, smart wristbands, etc.)) configured to sense a blood flow of the person. Digital (or photo) plesmography is optically detected blood volume changes in the microvascular bed of tissue. If the sensor is housed via a wrist band (or another wearable device) and worn on the person, then the sensor can sense the blood flow and thereby generate a waveform of the blood flow and communicate the waveform to a processor. In response, the processor can process the waveform and thereby predict, forecast, or estimate a virtual blood pressure (or heart rate) and a likely change in the virtual blood pressure (or heart rate) and hence request a repeat blood pressure (or heart rate) measurement for the person via an inflatable cuff only when an indication of a significant change in the virtual blood pressure (or heart rate) is sensed, where the indication satisfies or does not satisfy a preset or predetermined threshold for the virtual blood pressure (or heart rate) value. Therefore, this approach can significantly reduce an amount of blood pressure (or heart rate) measurements required from the person and therefore (1) be comfortable for the person when the person is conscious, (2) enable relaxation of the person, (3) allow the person to fall asleep, or (4) avoid waking the person if the person is already asleep.

This disclosure is now described more fully with reference to FIGS. 1-4, in which some embodiments of this disclosure are shown. This disclosure may, however, be embodied in many different forms and should not be construed as necessarily being limited to the embodiments disclosed herein. Rather, these embodiments are provided so that this disclosure is thorough and complete, and fully conveys various concepts of this disclosure to skilled artisans.

Various terminology used herein can imply direct or indirect, full or partial, temporary or permanent, action or inaction. For example, when an element is referred to as being “on,” “connected,” or “coupled” to another element, then the element can be directly on, connected, or coupled to another element or intervening elements can be present, including indirect or direct variants. In contrast, when an element is referred to as being “directly connected” or “directly coupled” to another element, then there are no intervening elements present.

As used herein, various singular forms “a,” “an” and “the” are intended to include various plural forms (e.g., two, three, four, five, six, seven, eight, nine, ten, tens, hundreds, thousands) as well, unless specific context clearly indicates otherwise.

As used herein, various presence verbs “comprises,” “includes” or “comprising,” “including” when used in this specification, specify a presence of stated features, integers, steps, operations, elements, or components, but do not preclude the presence or addition of one or more other features, integers, steps, operations, elements, components, or groups thereof.

As used herein, a term “or” is intended to mean an inclusive “or” rather than an exclusive “or.” That is, unless specified otherwise, or clear from context, “X employs A or B” is intended to mean any of a set of natural inclusive permutations. That is, if X employs A; X employs B; or X employs both A and B, then “X employs A or B” is satisfied under any of the foregoing instances.

As used herein, a term “or others,” “combination”, “combinatory,” or “combinations thereof” refers to all permutations and combinations of listed items preceding that term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. Skilled artisans understand that typically there is no limit on number of items or terms in any combination, unless otherwise apparent from the context.

As used herein, unless otherwise defined, all terms (including technical and scientific terms) used herein have the same meaning as commonly understood by one of ordinary skill in an art to which this disclosure belongs. Various terms, such as those defined in commonly used dictionaries, should be interpreted as having a meaning that is consistent with a meaning in a context of a relevant art and should not be interpreted in an idealized or overly formal sense unless expressly so defined herein.

As used herein, relative terms such as “below,” “lower,” “above,” and “upper” can be used herein to describe one element's relationship to another element as illustrated in the set of accompanying illustrative drawings. Such relative terms are intended to encompass different orientations of illustrated technologies in addition to an orientation depicted in the set of accompanying illustrative drawings. For example, if a device in the set of accompanying illustrative drawings were turned over, then various elements described as being on a “lower” side of other elements would then be oriented on “upper” sides of other elements. Similarly, if a device in one of illustrative figures were turned over, then various elements described as “below” or “beneath” other elements would then be oriented “above” other elements. Therefore, various example terms “below” and “lower” can encompass both an orientation of above and below.

As used herein, a term “about” or “substantially” refers to a +/−10% variation from a nominal value/term. Such variation is always included in any given value/term provided herein, whether or not such variation is specifically referred thereto.

Features described with respect to certain embodiments may be combined in or with various some embodiments in any permutational or combinatory manner. Different aspects or elements of example embodiments, as disclosed herein, may be combined in a similar manner.

Although the terms first, second, can be used herein to describe various elements, components, regions, layers, or sections, these elements, components, regions, layers, or sections should not necessarily be limited by such terms. These terms are used to distinguish one element, component, region, layer or section from another element, component, region, layer or section. Thus, a first element, component, region, layer, or section discussed below could be termed a second element, component, region, layer, or section without departing from various teachings of this disclosure.

Features described with respect to certain example embodiments can be combined and sub-combined in or with various other example embodiments. Also, different aspects or elements of example embodiments, as disclosed herein, can be combined and sub-combined in a similar manner as well. Further, some example embodiments, whether individually or collectively, can be components of a larger system, wherein other procedures can take precedence over or otherwise modify their application. Additionally, a number of steps can be required before, after, or concurrently with example embodiments, as disclosed herein. Note that any or all methods or processes, at least as disclosed herein, can be at least partially performed via at least one entity in any manner.

Example embodiments of this disclosure are described herein with reference to illustrations of idealized embodiments (and intermediate structures) of this disclosure. As such, variations from various illustrated shapes as a result, for example, of manufacturing techniques or tolerances, are to be expected. Thus, various example embodiments of this disclosure should not be construed as necessarily limited to various particular shapes of regions illustrated herein, but are to include deviations in shapes that result, for example, from manufacturing.

Any or all elements, as disclosed herein, can be formed from a same, structurally continuous piece, such as being unitary, or be separately manufactured or connected, such as being an assembly or modules. Any or all elements, as disclosed herein, can be manufactured via any manufacturing processes, whether additive manufacturing, subtractive manufacturing, or other any other types of manufacturing. For example, some manufacturing processes include three dimensional (3D) printing, laser cutting, computer numerical control routing, milling, pressing, stamping, vacuum forming, hydroforming, injection molding, lithography, and so forth.

FIG. 1 illustrates a schematic diagram of an embodiment including a processor in communication with a memory, a sensor, and a sphygmomanometer according to various principles of this disclosure. In particular, an system 100 includes a processor 102, a memory 104, a blood flow sensor 106, and a sphygmomanometer 108. The system 100 may be battery powered (e.g., a rechargeable battery, a lithium ion battery, a single use battery). However, the system 100 may be mains powered (e.g., via a power cord or a data cable plugged into a suitable source of electrical power).

The processor 102 includes a processing circuit, a digital circuit, an integrated circuit, an application specific integrated circuit, an application specific integrated processor, a microprocessor, a single core processor, a multicore processor, a graphics processing unit, a physics processing unit, a digital signal processor, a coprocessor, a network processor, a front-end processor, a field-programmable gate array, a programmable logic controller, a system-on-chip, or another form of processing logic. The processor 102 is in communication (e.g., wired, wireless, waveguide) with the memory 104, the blood flow sensor 106, and the sphygmomanometer 108 such that the processor 102 can control the memory 104, the blood flow sensor 106, and the sphygmomanometer 108 by sending and receiving data therefrom. Note that the blood flow sensor 106 or the sphygmomanometer 108 can include the processor 102. The processor 102 can be at least one of included in, physically secured to, electrically connected to, is a component of, or embodied as at least one of a hat, a helmet, a earbud, a hearing aid, a headphone, an eyewear frame, an eye lens, a band, a garment, a shoe, a jewelry item, a medical device, an activity tracker, a handcuff, a wristband, a smartphone, a tablet, a laptop, a desktop, a vehicle, an implant, a hat, a cap, a skullcap, a headband, a jacket, a shirt, a tie, a belt, a band, a pair of shorts, a pair of pants, a sock, an undershirt, an underwear item, a bra, a jersey, a skirt, a dress, a blouse, a sweater, a scarf, a glove, a bandana, an elbow pad, a kneepad, a pajama, a robe, a shoe, a dress shoe, a sneaker, a boot, or a heeled shoe.

The memory 104 includes a non-transitory medium that is configured to store data for usage by the processor 102. This data can be read, written, modified, or deleted by the processor 102. For example, the memory 104 can store a set of instructions executable by the processor 102 to perform various techniques, as disclosed herein.

The blood flow sensor 106 is configured to contact the person, whether positioned on an outer skin surface of the person (e.g., a limb, a torso, a head, a wrist, a finger, a toe, an upper arm) or over a material extending on the person (e.g., a bandage, a gauze pad, a garment). For example, the blood flow sensor 106 can be worn on the person as a wristband. For example, the blood flow sensor 106 can be an ultrasonic sensor, an ultrasonic Doppler sensor, a hermetically sealed Doppler probe, a continuous-wave sensor, a continuous-wave ultrasound Doppler blood flow sensor, an oxygen saturation sensor-pulse oximeter, or others (such as, photometric sensors (to obtain, for example, a photo (or digital) plesmographgram) on such things but not limited to (smartphones, smartwatches, tablets, smart wristbands, etc.)). For example, the blood flow sensor 106 can be a BlueDop® probe, as commercially available. When the blood flow sensor 106 contacts (or is close to contacting) the person, the blood flow sensor 106 senses a blood flow of the person and responsively generates a set of data representative of the blood flow. The set of data can be plotted to be visualized (e.g., via an oscilloscope) as a waveform of the blood flow (e.g., a volume of blood displacement as a function of time). For example, this set of data can be a plotted on a scale where an X-axis corresponds to a time interval (or distance) and a Y-axis corresponds to a volume of blood displaced. For example, this set of data can be similarly plotted as a force over distance, i.e., work. The movement of blood though blood vessels is work in the classical sense—force over a distance. Determining blood pressure based on the area under the curve, or “Work”. Work is the force over a distance, and force is mass times acceleration. If we say that the blood flow waveform (e.g., Doppler waveform) is the force curve with the summation of both forward movement of mass (blood) and the reverse movement of mass, then the waveform is a force curve of blood. Blood pressure being the force represented by the slope for the waveform per moment in time and the amplitude representing the mass (volume of blood) the distance is the visual spectrum of e.g., the Doppler probe. Thus the work is the summation of the area under the waveform. Now if we know the diameter of the pipe or vessel we can calculate a pressure based on Bernoulli's equation. But if we use the area under the curve and correlate that with a baseline pressure, i.e. proximal cuff pressure, we can follow changes in the area under the curve and calculate digital (i.e., virtual) blood pressure. This may be applied to any representative waveform of blood flow i.e. pulse ox waveform, pulse volume recording, Doppler, etc. But historically, utilizing this fact has been difficult because of factors such as the body changing the pipe (artery) diameter, the pipe (artery) stiffness (conservation of energy), and the compliance of the outflow vascular bed. For at least these reasons, trying to correlate heart rate to blood pressure gives very inaccurate results. Or trying to correlate an amplitude of a waveform by itself to blood pressure is only accurate for a single point in space and must be correlated to a blood pressure taken in the body at the same time so that the variable of artery diameter, stiffness, and outflow resistance is cancelled out. The inventors recognized that they can use the work of moving the blood, the area under the waveform, as a tool to estimate (calculate) and monitor blood pressure. The slope of the waveform, the duration to peak of the waveform, and other parameters are effected by the pipe diameter, stiffness, and outflow resistance. By considering properties of these parameters, we can examine the work of moving blood and from this we are able to follow blood pressure continuously for an extended period of time after initial calibration or measurement.

The blood flow sensor 106 functions as a waveform generator forming the waveform of the blood flow as the set of data and sending (e.g., wired, wireless, waveguide) the set of data to the processor 102, whether continuously or periodically, as requested by the processor 102.

The sphygmomanometer 108 includes a wearable cuff (e.g., a wrist cuff, an arm cuff), a bladder, a pump (e.g., a gas pump, an air pump), a valve (e.g., a solenoid valve, a gas valve, an air valve), a sensor (e.g., a pressure sensor, a transducer), and a controller, which can be the processor 102 or another processing device. The controller is in communication (e.g., wired, wireless, waveguide) with the pump, the valve, and the sensor such that the controller can control the pump, the valve, and the sensor by sending and receiving data therefrom. The wearable cuff includes the bladder. The valve can be included in the wearable cuff or excluded from the wearable cuff. As instructed by the controller, the pump is configured to pump a gas (e.g., a volume of air) into the bladder to inflate the wearable cuff, while the wearable cuff is worn on the person. The pump may also be inflated via a fluid. As instructed by the controller, the valve is configured to output the gas/fluid from the bladder such that the bladder deflates, while the wearable cuff is worn on the person. The sensor is configured to determine a systolic blood pressure measurement of the person and a diastolic blood pressure measurement of the person, while the bladder is being deflated via the valve, as instructed by the controller. The sensor sends (e.g., wired, wireless, waveguide) the systolic blood pressure measurement and the diastolic blood pressure measurement to the controller.

Note that the sphygmomanometer 108 may include an amplifier and an analog-to-digital converter (ADC). As such, the sensor may generate an analog signal (e.g., pressure readings) while the wearable cuff is worn on the person. The sensor may send the analog signal to the amplifier for amplification. The amplifier may send the analog signal, as amplified, to the ADC for conversion into a digital signal by the ADC. The ADC may send the digital signal to the controller which performs various local processing to determine the systolic pressure measurement of the person and the diastolic pressure measurement of the person, which may be along with a pulse rate of the person.

The sphygmomanometer 108 can include at least one of the processor 102, the memory 104, or the blood flow sensor 106. For example, the sphygmomanometer 108 can include a housing containing the processor 102 and the memory 104, while the blood flow sensor 106 is worn on the person external to the housing. For example, the wearable cuff can include the blood flow sensor 106 or the wearable cuff can exclude the blood flow sensor 106 (e.g., the wearable cuff is worn on the upper arm of the person and the blood flow sensor 106 is included in the wristband worn on the wrist of the person, or vice versa, or on a finger cuff worn on the finger of the person). However, note that the blood flow sensor 106 can be embodied in other ways or non-invasively worn in other areas of the person (e.g., a limb, a head, a torso, a finger, a toe, an upper arm). In some implementations, the sphygmomanometer 108 is omitted and the processor 102 receives the systolic blood pressure measurement of the person and the diastolic blood pressure measurement of the person from a wearable device (e.g., an activity tracker, a wristband activity tracker, a smartwatch, smartphones), which may itself obtain the systolic blood pressure measurement of the person and the diastolic blood pressure measurement of the person when the wearable device is worn on the person, or receive the systolic blood pressure measurement of the person and the diastolic blood pressure measurement of the person from another device worn on the person or as manually input via the person or a caregiver of the person (e.g., a physician, a nurse, a guardian). Note that the diastolic blood pressure measurement of the person may be calculated, estimated, or forecasted from the systolic blood pressure measurement or be manually input via the person or a caregiver of the person (e.g., a physician, a nurse, a guardian).

In one mode of operation, the processor 102 may be programmed to request the sphygmomanometer 108 (or another device) to send (e.g., wired, wireless, waveguide) a first blood pressure measurement of the person (e.g., a first systolic blood pressure measurement and a first diastolic blood pressure measurement) at a first time instance to the processor 102. Likewise, the processor 102 may be programmed to request the blood flow sensor 106 to send (e.g., wired, wireless, waveguide) the set of data (forming the waveform) to the processor 102, where the set of data is generated by the blood flow sensor 106 based on the blood flow sensor 106 monitoring, whether continuously or periodically, the blood flow of the person, whether where the wearable cuff is worn on the person or at another site of the person, whether before, during, or after the sphygmomanometer 108 obtains the first blood pressure measurement or sends the first blood pressure measurement to the processor 102. As such, the processor 102 may be programmed to continuously analyze the set of data (forming the waveform) received from the blood flow sensor 106 and to determine whether there is a change in a waveform shape which would indicate a significant change in a blood pressure of the person, as determined based on a satisfaction or non-satisfaction of a preset or predetermined threshold. Accordingly, the processor 102 may be programmed to request the sphygmomanometer 108 to send (e.g., wired, wireless, waveguide) a second blood pressure measurement of the person (e.g., a second systolic blood pressure measurement and a second diastolic blood pressure measurement) at a second time instance, after the first time instance, to the processor 102 based on the processor 102 determining that there is the change in the waveform shape indicative of the significant change in the blood pressure of the person, as determined based on the satisfaction or non-satisfaction of the preset or predetermined threshold, or when a preset or predetermined time has passed since the first blood pressure measurement (e.g., about 4 to about 8 hours although each of these bounds can be varied). The first blood pressure measurement can be different from the first blood measurement.

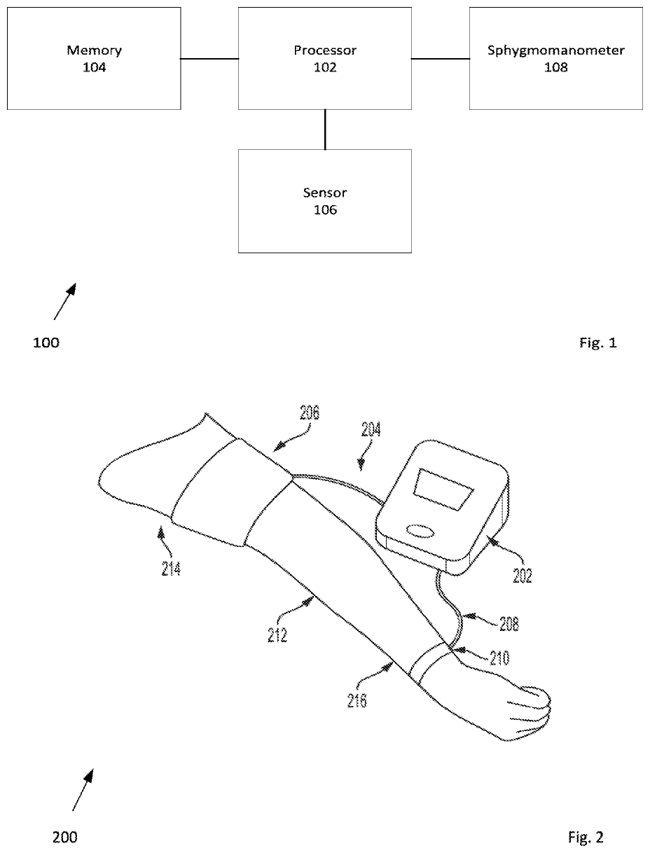

FIG. 2 illustrates a 3-dimensional diagram of an embodiment of a device configured for measuring a blood pressure of a patient pursuant to FIG. 1 according to various principles of this disclosure. In particular, an system 200 includes a housing 202, a tube 204 (e.g., a hose), a pressure cuff 206, a data line 208, and a blood flow sensor 210 (e.g., Doppler flow sensor). The pressure cuff 206 is worn on an upper arm 214 of an arm 212 of the person. The blood flow sensor 210 is worn on a wrist 216 of the arm 212.

The sphygmomanometer 108 includes the housing 202, the tube 204, the pressure cuff 206, the processor 102, and the memory 104. The housing 202 includes the pump of the sphygmomanometer 108, the valve of the sphygmomanometer 108, and the sensor of the sphygmomanometer 108, as described above. In some implementations, the housing 202 can include the amplifier and the analog-to-digital converter (ADC). The tube 204 spans between the pressure cuff 206 and the housing 202 such that the pump can inflate the pressure cuff 206 (e.g., the bladder) with a gas (e.g., a volume of air) or fluid pumped by the pump through the tube 204, as instructed via the controller, which can include the processor 102. The valve can output the gas/fluid from the pressure cuff 206 such that the pressure cuff 206 deflates, as instructed via the controller, which can include the processor 102. The sensor within the housing 202 can send its readings to the controller, which can be the processor 102, such that the controller can determine the systolic pressure measurement of the person and the diastolic pressure measurement of the person, or the sensor itself determines the systolic pressure measurement of the person and the diastolic pressure measurement of the person and sends the systolic pressure measurement of the person and the diastolic pressure measurement of the person to the controller.

The data line 208 (e.g., a data cable, a fiber optic cable) spans between the blood flow sensor 210 and the housing 202 such that the blood flow sensor 210 can send its data over the data line 208 to the controller (or vice versa), which can be the processor 102. In some implementations, the data line 208 is absent and each of the housing 202 and the blood flow sensor 210 includes a wireless networking interface (e.g., a wireless receiver, a wireless transmitter, a wireless transceiver) such that each respective network interface can communicate with each other. For example, the processor 102 can be in communication with the wireless networking interface of the housing 202 to send or receive data therefrom. As such, the blood flow sensor 210 (or other suitable sensors configured to sense a blood flow that arises from the heart of the person contracting as disclosed herein) can send its data wirelessly via its wireless networking interface to the housing 202 via its wireless networking interface, which in turn sends that data to the controller, which can be the processor 102. In some implementations, the blood flow sensor 210 is included in the pressure cuff 206. As such, the tube 204 can include the data line 208, whether internally or externally, or the data line 208 is omitted and the blood flow sensor 210 or the pressure cuff 206 can include the wireless networking interface to communicate with the wireless networking interface of the housing 202.

The housing 202 contains the processor 102, while the housing 202 is not worn on the person and is separate and distinct from each of the pressure cuff 206 and the blood flow sensor 210. However, note that this configuration can vary. For example, the pressure cuff 206 can include the housing 202 or the blood flow sensor 210. Likewise, the blood flow sensor 210 can include the housing 202 or the pressure cuff 206. Similarly, the housing 202 can include the blood flow sensor 210. Moreover, the processor 102 can be in at least one of wired or waveguide communication with the blood flow sensor 210, although wireless communication with the blood flow sensor 210 is possible. Note that the housing 202 includes a display (e.g., a digital display, an analog display). However, the housing 202 may also avoid the display.

In one mode of operation, the pressure cuff 206 is worn on the upper arm 214 and the blood flow sensor 210 is worn on the wrist 216. During this time, the processor 102 within the housing 202 may be programmed to request the pump within the housing 202 to inflate the pressure cuff 206 through the tube 204 and the valve within the housing 202 to deflate the pressure cuff 206 through the tube 204. Based on this inflation of the pressure cuff 206 and deflation of the pressure cuff 206, the sensor within the housing 202 generates a first set of pressure readings of the upper arm 214 and sends the first set of pressure readings to the processor 102 such that the processor 102 generates a first blood pressure measurement of the person (e.g., a first systolic blood pressure measurement and a first diastolic blood pressure measurement) at a first time instance based on the first set of readings. Likewise, the processor 102 may be programmed to request the blood flow sensor 210 to send the set of data (forming the waveform) to the processor 102 over the data line 208, where the set of data is generated by the blood flow sensor 210 based on the blood flow sensor 210 monitoring, whether continuously or periodically, the blood flow of the person, whether before, during, or after the processor 102 obtains the first blood pressure measurement. As such, the processor 102 may be programmed to continuously analyze the set of data (forming the waveform) received from the blood flow sensor 210 and to determine whether there is a change in a waveform shape which would indicate a significant change in a blood pressure of the person, as determined based on a satisfaction or non-satisfaction of a preset or predetermined threshold. Accordingly, the processor 102 may be programmed to request the pump within the housing 202 to inflate the pressure cuff 206 through the tube 204 and the valve within the housing 202 to deflate the pressure cuff 206 through the tube 204 responsive to the processor 102 determining that there is the change in the waveform shape indicative of the significant change in the blood pressure of the person, as determined based on the satisfaction or non-satisfaction of the preset or predetermined threshold, or when a preset or predetermined condition, for example time period, has passed since the first blood pressure measurement (e.g., about 4 to about 8 hours although each of these bounds can be varied). Based on this inflation of the pressure cuff 206 and deflation of the pressure cuff 206, the sensor within the housing 202 generates a second set of pressure readings of the upper arm 214 and sends the second set of pressure readings to the processor 102 such that the processor 102 generates a second blood pressure measurement of the person (e.g., a second systolic blood pressure measurement and a second diastolic blood pressure measurement) at a second time instance, after the first time instance, based on the second set of readings. The first blood pressure measurement can be different from the first blood measurement.

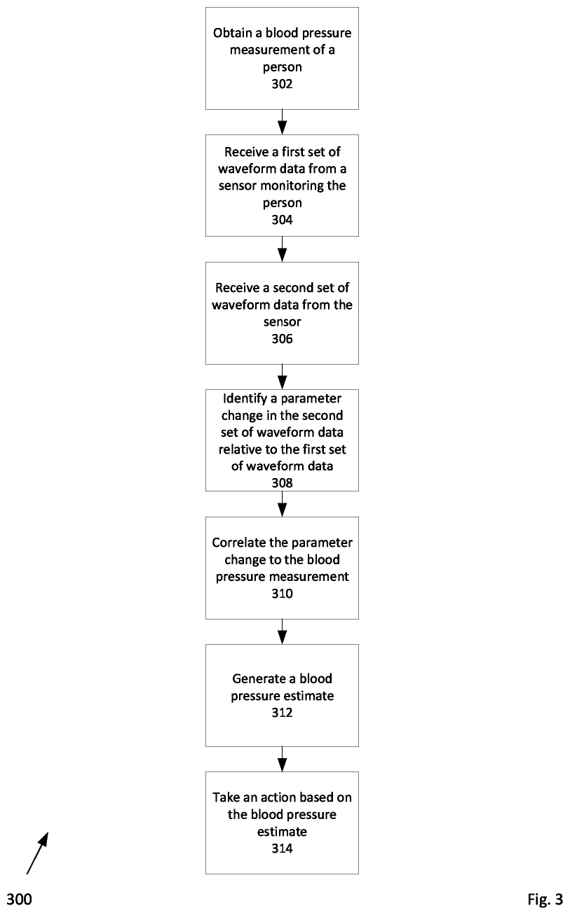

FIG. 3 illustrates a flowchart of an embodiment of a process for generating a virtual blood pressure (or heart rate) (as used throughout this disclosure, a virtual blood pressure is a calculated value based on a blood pressure measurement and parameters of the waveform data sets) pursuant to FIGS. 1-2 according to various principles of this disclosure. In particular, a process 300 includes a set of blocks 302-314 performed via the system 100 of FIG. 1 or the system 200 of FIG. 2.

In block 302, the processor 102 obtains a blood pressure measurement (e.g., a systolic blood pressure measurement and a diastolic blood pressure measurement) of a person. The processor 102 forms the blood pressure measurement from a set of readings received from the sensor housed within the housing 202 of the sphygmomanometer 108. Alternatively, the processor 102 can receive the blood pressure measurement from the sensor housed within the housing 202 of the sphygmomanometer 108. Or, the processor 102 can receive the blood pressure measurement from another device (e.g., an activity tracker, a wristband activity tracker, a smartwatch, smartphones), which may itself obtain the blood pressure measurement when the wearable device is worn on the person or receive the blood pressure measurement from another device worn on the person or as manually input via the person or a caregiver of the person (e.g., a physician, a nurse, a guardian).

In block 304, the processor 102 receives (e.g., wired, wireless, waveguide) a first set of waveform data from a sensor (e.g., the blood flow sensor 106, the Doppler flow sensor 210, a continuous-wave sensor) monitoring the person. This can occur via the processor 102 instructing the sensor worn on the person to monitor a first blood flow of the person during a time period (e.g., less than, equal to, or greater than about 1, 2, 3, 4, 5, 6, 7, 8, 9, 10 seconds, 1, 2, 3, 4, 5, 6, 7, 8, 9, 10 minutes) including a cardiac cycle such that the sensor generates the first set of waveform data representing the cardiac cycle during the time period. The first set of waveform data is associated with the blood pressure measurement. For example, the blood pressure measurement can be taken before, during, or after the first set of data is generated by the sensor. When the person has a head, a torso, a limb, a wrist, a hand, a finger, an ankle, a toe, or a thigh, the sensor can be worn on at least one of the head, the torso, the limb, the wrist, the hand, the finger, the ankle, the toe, or the thigh. However, note that the blood pressure monitor (e.g., the sphygmomanometer 108) can include the pressure cuff 206 worn by the person (e.g., the upper arm 214, the wrist 216) where the pressure cuff 206 enables the blood pressure measurement (e.g., via inflation and deflation thereof) and the pressure cuff 206 can include the sensor, whether internally or externally.

In block 306, the processor 102 receives (e.g., wired, wireless, waveguide) a second set of waveform data for a second blood flow from the sensor after the first set of waveform data is generated by the sensor.

In block 308, the processor 102 identifies a parameter change in the second set of waveform data relative to the first set of waveform data. This can occur via the processor 102 reading the first set of waveform data and the second set of waveform data, comparing the second set of waveform data against the first set of waveform data (e.g., based on values at specific time points), and identifying a difference of the second set of waveform data relative to the first set of waveform data, where the difference is informative of the parameter change. For example, the difference can be based on values at specific time points or a range of values for a range of time points. For example, the processor 102 can be programmed to identify the parameter change based on deriving a first parameter of a first waveform shape of the first set of waveform data for each cardiac cycle during the first blood flow, deriving a second parameter of a second waveform shape of the second set of waveform data for each cardiac cycle during the second blood flow, and identifying a change between the first parameter and the second parameter. For example, each of the first parameter and the second parameter is a heartbeat period. For example, each of the first parameter and the second parameter is a height of an initial upswing, as further described below. For example, each of the first parameter and the second parameter is a period of an initial upswing, as further described below. For example, each of the first parameter and the second parameter is a frequency of an oscillatory component, as further described below.

In block 310, the processor 102 correlates the parameter change to the blood pressure measurement, as further described below. For example, the parameter change can be a change in a waveform shape.

In block 312, the processor 102 generates a virtual blood pressure (or heart rate) (a calculated value based on the blood pressure measurement and parameters of the waveform data sets) for the second blood flow, as further described below.

In block 314, the processor 102 takes an action based on the virtual blood pressure (or heart rate). For example, the action can include instructing a blood pressure monitor (e.g., the sphygmomanometer 108) to obtain another blood pressure measurement that is different from the blood pressure measurement, as associated with the first set of waveform data. Note that the blood pressure measurement, as associated with the first set of waveform data, can be taken by the blood pressure monitor as well. For example, the action can include requesting or instructing an input device (e.g., a touchscreen, a microphone, a camera, a sensor) to request, instruct, read, receive, generate, input, prompt, or open for an input, which can include a user input, whether from the person wearing the sensor or a caretaker thereof. For example, the action can include requesting or instructing an output device (e.g., a display, a speaker, a sensor, a motor, a pump, a valve) to request, instruct, read, receive, transmit, generate, output, prompt, or open for an output, which can include an output to a user, whether to the person wearing the sensor or a caretaker thereof. For example, the action can include the processor 102 determining if the virtual blood pressure (or heart rate) is within a predetermined range or percentage thereof for an actually obtained blood pressure of the person. If within the predetermined range or percentage thereof, then no new calibration (e.g., the blood flow sensor 106, the Doppler flow sensor 210, a continuous-wave sensor) needs to occur. However, if outside the predetermined range or percentage thereof then recalibration (e.g., the blood flow sensor 106, the Doppler flow sensor 210, a continuous-wave sensor) will occur. For example, the action can include instructing an output device (e.g., a display, a speaker) or a transmitter (e.g., wired, wireless, waveguide) to respectively output or transmit the virtual blood pressure or a virtual heart rate, as disclosed herein.

In one mode of operation, the person can be at rest and the processor 102 can be programmed to generate a first blood pressure (or heart rate) measurement based on a first set of readings from a pressure sensor (e.g., housed within the housing 202) when the pressure cuff 206 worn on the person is inflated and then deflated, as disclosed herein. In some implementations, the first blood pressure (or heart rate) measurement is manually entered into the processor 102 (e.g., via an input device like a touchscreen, a cursor device, a microphone or another suitable input device) or otherwise availed to the processor 102 or received from another device (e.g., an activity tracker, a ring, finger clip, a wristband activity tracker, a smartwatch, a blood pressure monitor, photometric sensors (to obtain, for example, a photo (or digital) plesmographgram) on such things but not limited to (smartphones, smartwatches, tablets, smart wristbands, etc.), which may itself obtain the blood pressure (or heart rate) measurement when the wearable device is worn on the person or receive the blood pressure (or heart rate) measurement from another device worn on the person or as manually input via the person or a caregiver of the person (e.g., a physician, a nurse, a guardian).

The processor 102 can be programmed to instruct a sensor (e.g., continuous wave sensor, the Doppler flow sensor 210) worn on the person (e.g., the wrist 216) to monitor in real-time a first blood flow of the person during a first time period (e.g., less than, equal to, or greater than about 1, 2, 3, 4, 5, 6, 7, 8, 9 10, tens, hundreds, thousands seconds, minutes, hours including any whole or partial intermediary values) including a first cardiac cycle such that the sensor generates a first set of readings representing the first cardiac cycle during the first time period. The first set of readings is associated with the first blood pressure (or heart rate) measurement and each reading of the first set of readings includes a first data value and a first time value. For example, the first blood pressure (or heart rate) measurement can before, during, or after the sensor generates the first set of readings or vice versa. The first set of readings can be obtained before the first blood pressure (or heart rate) measurement. For example, the first set of readings can be obtained before the first blood pressure (or heart rate) measurement within a predetermined time period immediately before the processor 102 determines the first blood pressure (or heart rate) measurement or receives the first blood pressure (or heart rate) measurement from another device (e.g., the pressure sensor, wearable). For example, the predetermined time period can be about 1 hour or less or more. However, note that the first set of readings can be obtained concurrent with the first blood pressure (or heart rate) measurement or obtained after the first blood pressure (or heart rate) measurement.

The processor 102 can be programmed to instruct the sensor worn on the person to monitor in real-time a second blood flow of the person during a second time period (e.g., less than, equal to, or greater than about 1, 2, 3, 4, 5, 6, 7, 8, 9 10, tens, hundreds, thousands seconds, minutes, hours including any whole or partial intermediary values) including a second cardiac cycle such that the sensor generates a second set of readings representing the second cardiac cycle during the second time period. The first time period may be identical or not be identical to the second time period, whether overlapping or not overlapping. Each reading of the second set of readings includes a second data value and a second time value and the second time period begins after the first time period begins. The first time period and the second time period can overlap with each other or avoid overlapping with each other. The second time period can consecutively follow the first time period or there may be a third time period intervening between the first time period and the second time period. The first cardiac cycle and the second cardiac cycle can be a same cardiac cycle or different cardiac cycles.

The processor 102 can be programmed to populate a data structure (e.g., a two-dimensional data structure, a three-dimensional data structure, a multi-dimensional data structure, an array, a vector) with the first set of readings and the second set of readings such that the first set of readings can plot a first blood flow waveform corresponding to the first set of readings based on the first data values and the first time values and the second set of readings can plot a second blood flow waveform corresponding to the second set of readings based on the second data values and the second time values, as disclosed herein. The processor 102 can be programmed to populate the data structure with the first set of readings and the second set of readings when the sensor (e.g. continuous wave sensor) contacts (or is close to contacting) the person or is worn on the person, as disclosed herein.

The processor 102 can be programmed to derive a first set of parameters from the first set of readings including the first data values and the first time values and to derive a second set of parameters from the second set of readings including the second data value and the second time values, as disclosed herein. The processor 102 can be programmed to perform a comparison between the first set of parameters and the second set of parameters and determine whether the second set of parameters is indicative of a predetermined change in the second blood flow waveform relative to the first blood flow waveform based on the comparison, where the predetermined change is indicative of a value change in the first blood pressure measurement. For example, the value change can be the blood pressure (or heart rate) measurement being higher or lower than previously measured. For example, the value change or the predetermined change can satisfy or not satisfy a predetermined threshold. The processor 102 can be programmed to derive the first set of parameters from the first set of readings including the first data values and the first time values when the sensor contacts the person or is worn on the person, as disclosed herein. The processor 102 can be programmed to derive the second set of parameters from the second set of readings including the second data value and the second time values when the sensor contacts the person or is worn on the person, as disclosed herein. The processor 102 can be programmed to perform the comparison between the first set of parameters and the second set of parameters when the sensor contacts the person or is worn on the person, as disclosed herein.

The processor 102 can be programmed to generate a second blood pressure measurement based on a second set of readings from the pressure sensor (e.g., housed within the housing 202) when the pressure cuff 206 worn on the person is inflated and then deflated, as disclosed herein. The processor 102 generates the second blood pressure (or heart rate) measurement based on at least one of the predetermined change satisfying a predetermined threshold for the person or a preset time period (e.g., about 24, 23, 22, 21, 20, 19, 18, 17, 16, 15, 14, 13, 12, 11, 10, 9, 8, 7, 6, 5, 4, 3, 2, or 1 hour or less including any intermediary whole or decimal values) having expired from when the first blood (or heart rate) measurement was generated, where the second blood pressure (or heart rate) measurement is after the first blood pressure (or heart rate) measurement. The processor 102 can be programmed to derive the first set of parameters or the second set of parameters based on a mathematical function or a result of the mathematical function. For example, the mathematical function can be a mean function, a median function, a mode function, or another mathematical function. Note that each modality of derivation can be identical or non-identical in terms of the mathematical function. The processor 102 can be programmed to determine whether the second set of parameters is indicative of the predetermined change in the second blood flow waveform relative to the first blood flow waveform based on the comparison when the continuous-wave sensor contacts the person or is worn on the person, as disclosed herein. The processor 102 can be programmed to generate the second blood pressure (or heart rate) measurement or receive the second blood pressure (or heart rate) measurement from the pressure sensor (or another device) when the pressure cuff 206 is worn on the person when the sensor contacts the person or is worn on the person, as disclosed herein.

When a person with an arm 212, the pressure cuff 206 and the sensor can be worn on the arm 212 (e.g., at the wrist 216 or finger, at the upper arm 214, at both). However, note that such configuration can vary. For example, the person can have a first body part (e.g., a head, a torso, a limb, the arm 212, the upper arm 214, the wrist 216, a finger, a toe, a thigh, an ankle, a leg or any part thereof) and a second body part (e.g., a head, a torso, a limb, the arm 212, the upper arm 214, the wrist 216, a finger, a toe, a thigh, an ankle, a leg or any part thereof), where the pressure cuff 206 can be worn on the first body part and the sensor is worn on the second body part, whether same or different body parts. In other examples, the pressure cuff 206 can include the sensor.

As further explained below, the first blood flow waveform has a first shape when displayed (e.g., via an oscilloscope) and the second blood flow waveform has a second shape when displayed (e.g., via an oscilloscope). As such, the processor 102 can be programmed to determine whether the second set of parameters is indicative of the predetermined change in the second blood flow waveform relative to the first blood flow waveform based on the comparison involving a shape change between the first shape and the second shape, where the shape change corresponds to the value change in the first blood pressure (or heart rate) measurement. The first set of parameters can include a first parameter corresponding to a first time period of the first cardiac cycle and the second set of parameters can include a second parameter corresponding to a second time period of the second cardiac cycle, where the first shape corresponds to the first time period, wherein the second shape corresponds to the second time period. The first set of parameters can include a first parameter corresponding to a first height value of a first initial upswing of the first cardiac cycle and the second set of parameters can include a second parameter corresponding to a second height value of a second initial upswing of the second cardiac cycle, where the first shape corresponds to the first height value, wherein the second shape corresponds to the second height value. The first set of parameters can include a first parameter corresponding to a first time period of a first initial upswing of the first cardiac cycle and the second set of parameters can include a second parameter corresponding to a second time period value of a second initial upswing of the second cardiac cycle, where the first shape corresponds to the first time period and the second shape corresponds to the second time period. The first set of parameters can include a first parameter corresponding to a first frequency of a first oscillatory component of the first cardiac cycle and the second set of parameters can include a second parameter corresponding to a second frequency of a second oscillatory component of the second cardiac cycle, where the first shape corresponds to the first frequency of the first oscillatory component and the second shape corresponds to the second frequency of the second oscillator component. The comparison includes comparing the first set of parameters against the second set of parameters or vice versa. As such, the processor 102 can be programmed to determine in real-time whether the second set of parameters is indicative of the predetermined change in the second blood flow waveform relative to the first blood flow waveform based on the comparison.

As disclosed herein, the processor 102 can be programmed to instruct the sensor contacting the person (e.g., an outer skin surface, a bandage, a gauze pad, a garment) or worn on the person (e.g., a head, a torso, a limb, the arm 212, the upper arm 214, the wrist 216, a finger, a toe, a thigh, an ankle, a leg or any part thereof) to monitor in real-time the first blood flow of the person during the first time period including a set of cardiac cycles (e.g., 1, 2, 3, 4, 5, 6, 7, 8, 9, 10, tens, hundreds, thousands including any intermediary whole values) including the first cardiac cycle such that the sensor generates the first set of readings representing the set of cardiac cycles during the first time period. Likewise, the processor 102 can be programmed to instruct the sensor contacting the person (e.g., an outer skin surface, a bandage, a gauze pad, a garment) or worn on the person (e.g., a head, a torso, a limb, the arm 212, the upper arm 214, the wrist 216, a finger, a toe, a thigh, an ankle, a leg or any part thereof) to monitor in real-time the second blood flow of the person during the second time period including a set of cardiac cycles including the second cardiac cycle such that the sensor generates the second set of readings representing the set of cardiac cycles during the second time period, whether worn on same or different body parts. The set of cardiac cycles during the first time period and the set of cardiac cycles during the second time period can be overlapping or not overlapping, whether consecutive or non-consecutive, whether same ones or different ones. The first cardiac cycle can be included in the second time period or the second cardiac cycle can be included in the first time period. The first time period and the second time period can be consecutive or not consecutive, overlap with each other or not overlap with each other. The first time period and the second time period can be spaced apart from each other by a third time period (e.g., intervening time period). The third time period can be about 24, 23, 22, 21, 20, 19, 18, 17, 16, 15, 14, 13, 12, 11, 10, 9, 8, 7, 6, 5, 4, 3, 2, or 1 hour or less including any intermediary whole or decimal values.

As disclosed herein, the processor 102 can be programmed to instruct the sensor worn on the patient to monitor in real-time the first blood flow of the patient during the first time period including the first cardiac cycle such that the sensor generates the first set of readings cumulatively representing the first cardiac cycle during the first time period as at least one of a mathematical function or a result obtained from the mathematical function. For example, the mathematical function can be a mean function, a median function, a mode function, or another mathematical function. Likewise, the processor 102 can be programmed to instruct the sensor worn on the patient to monitor in real-time the second blood flow of the patient during the second time period including the second cardiac cycle such that the sensor generates the second set of readings cumulatively representing the second cardiac cycle during the first time period as a mathematical function or a result obtained from the mathematical function. For example, the mathematical function can be a mean function, a median function, a mode function, or another mathematical function. Note that the mathematical function for the first set of readings and the mathematical functions for the second set of readings can be an identical mathematical function or a non-identical mathematical function.

As disclosed herein, the processor 102 can be programmed to populate the data structure with the first set of readings before the second set of readings is generated by the sensor or received by the processor 102, concurrent with the second set of readings being generated by the sensor or received by the processor 102 or after the second set of readings is generated by the sensor or received by the processor 102. The first blood flow waveform can be plotted on at least one of a Cartesian plane or an x-y plane based on the first data values and the first time values. The second blood flow waveform can be plotted on at least one of a Cartesian plane or an x-y plane based on the second data values and the second time values.

As disclosed herein, there can be a system comprising the processor 102 and a sensor (e.g., the blood flow sensor 106, the Doppler flow sensor 210). The sensor is configured to contact the person (e.g., the arm 212, the upper arm 214, the wrist 216, a thigh, an angle, a leg or any part thereof) or to be worn on a person (e.g., the arm 212, the upper arm 214, the wrist 216, a thigh, an angle, a leg or any part thereof), where the processor 102 is in communication with the sensor. The processor 102 can be programmed to instruct the sensor to monitor in real-time a blood flow of the person when the sensor is worn on the person while the blood flow is associated with a first blood pressure (or heart rate) measurement (e.g., via the sphygmomanometer 108 or another device). The sensor generates a waveform associated with the blood flow and sends the waveform to the processor 102. The processor 102 can be programmed to determine whether there is a shape change in the waveform indicative of a value change in the first blood pressure (or heart rate) measurement and obtain a second blood pressure (or heart rate) measurement of the person based on at least one of the shape change satisfying a predetermined threshold for the patient or a preset time period having expired from when the first blood (or heart rate) measurement was received, where the second blood pressure measurement is after the first blood pressure measurement.

As disclosed herein, there can be a system comprising the processor 102 programmed to instruct a sensor (e.g., the blood flow sensor 106, the Doppler flow sensor 210) configured to contact the person (e.g., the arm 212, the upper arm 214, the wrist 216, a thigh, an angle, a leg or any part thereof) or to be worn on the person (e.g., the arm 212, the upper arm 214, the wrist 216, a thigh, an angle, a leg or any part thereof) to monitor a baseline blood flow of the patient during a baseline time period including a cardiac cycle such that the sensor generates a set of readings representing the cardiac cycle during the baseline time period. The set of readings is associated with a baseline blood (or heart rate) pressure measurement (e.g., via the sphygmomanometer 108 or another device) wherein the cardiac cycle is concurrent to the baseline blood pressure measurement. The processor 102 can be programmed to receive a set of continuous waveform data for a non-baseline blood flow from the sensor after the set of readings is generated by the sensor, identify a parameter change in the set of continuous waveform data; correlate the parameter change to the baseline blood pressure (or heart rate) measurement such that a virtual blood pressure (or heart rate) for the non-baseline blood flow is generated; and take an action based on the virtual blood pressure. For example, the action can include obtaining a non-baseline blood pressure measurement or instructing another device to do something (e.g., write data, modify data, erase data, activate a physical device like a pump, an actuator, a motor or another physical device).

As disclosed herein, there can be a system comprising a processor 102, an input device (e.g., the sphygmomanometer 108, a wearable device, a remote device), and a sensor (e.g., the blood flow sensor 106, the Doppler flow sensor 210), where the sensor is configured to contact the person (e.g., the arm 212, the upper arm 214, the wrist 216, a thigh, an angle, a leg or any part thereof) or to be worn on the person (e.g., the arm 212, the upper arm 214, the wrist 216, a thigh, an angle, a leg or any part thereof). The processor 102 is in communication with the input device and the sensor. The processor 102 can be programmed to: instruct the sensor to monitor a current blood flow of the person when the sensor contacts the person or is worn on the person, as disclosed herein, while the current blood flow is associated with a current blood pressure measurement received from the input device such that the sensor generates a waveform associated with the current blood flow. The processor 102 can be programmed to determine whether there is a shape change in the waveform indicative of a value change in the blood pressure (or heart rate) measurement, as disclosed herein. The processor 102 can be programmed to take an action based on the shape change satisfying a predetermined threshold for the patient. For example, the action can include obtaining a non-baseline blood pressure measurement or instructing another device to do something (e.g., write data, modify data, erase data, activate a physical device like a pump, an actuator, a motor or another physical device).

As disclosed herein, there can be a device comprising a processor 102 programmed to: instruct a sensor (e.g., the blood flow sensor 106, the Doppler flow sensor 210), where the sensor is configured to contact the person (e.g., the arm 212, the upper arm 214, the wrist 216, a thigh, an angle, a leg or any part thereof) or to be worn on the person (e.g., the arm 212, the upper arm 214, the wrist 216, a thigh, an angle, a leg or any part thereof) to monitor a baseline blood flow of the patient during a baseline time period including a cardiac cycle such that the sensor generates a set of readings representing the cardiac cycle during the baseline time period. The set of readings is associated with a baseline blood pressure measurement and the cardiac cycle is concurrent to, simultaneous with, immediately before, immediately after, or otherwise associated with the baseline blood pressure (or heart rate) measurement. The processor 102 can be programmed to receive a set of continuous waveform data for a non-baseline blood flow from the sensor after the set of readings is generated by the sensor; identify a parameter change in the set of continuous waveform data; correlate the parameter change to the baseline blood pressure measurement such that a virtual blood pressure (or heart rate) for the non-baseline blood flow is generated; and take an action based on the virtual blood pressure, as disclosed herein. For example, the action can include obtaining a non-baseline blood pressure measurement or instructing another device to do something (e.g., write data, modify data, erase data, activate a physical device like a pump, an actuator, a motor or another physical device).

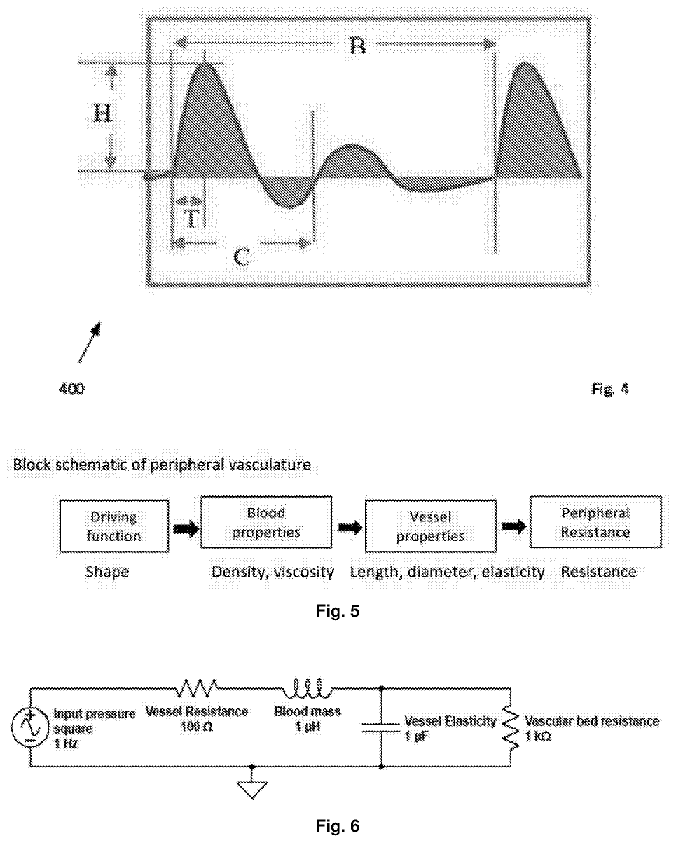

FIG. 4 illustrates a wave diagram of an embodiment of a blood flow waveform pursuant to FIGS. 1-3 according to various principles of this disclosure. In particular, the system 100 or the system 200 can perform the method 300 based on a wave diagram 400. The wave diagram 400 has an X-axis representative of time and a Y-axis representative of a displacement of a volume of blood of a person, while the person is wearing a sensor (e.g., the blood flow sensor 106, the Doppler flow sensor 210), as disclosed herein.

Conventionally, an invasive monitoring of blood pressure is usually performed by an indwelling arterial catheter that uses a fluid column to measure blood pressure. Likewise, a non-invasive monitoring of blood pressure is usually performed by frequent repeat measurements using the pressure cuff 206 worn on the upper arm 214 of the person. The pressure cuff 206 is inflated to a pressure above a maximum blood pressure expected for the person, and then slowly deflated while sensing for pulses in pressure within the pressure cuff 206 to obtain the systolic blood pressure measurement (maximum) and the diastolic pressure measurement (minimum) in some underlying arterial vessels (this is known as the oscillometric method). This process is usually repeated to provide frequent updates and this frequent repetition may be found uncomfortable for conscious patients and prevent relaxation and the ability to fall asleep. As such, by using an additional blood flow detector (e.g., the continuous-wave sensor, the blood flow sensor 106, the Doppler flow sensor 210) in a wrist band (or another wearable form factor) or physiologic blood flow derived, a blood flow waveform can be generated by the additional blood flow detector and used by a processor to generate a virtual blood pressure and to predict likely changes in blood pressure and hence require a repeat pressure cuff measurement only when an indication of significant change is sensed, predicted, forecast, or estimated. This approach will significantly reduce a number of measurements (e.g., blood pressure, heart rate) via the pressure cuff 206 required and therefore reduce patient discomfort, as disclosed herein. For example, initially, a brachial pressure cuff device (e.g., the sphygmomanometer 108) can be used to measure systolic and diastolic pressure in the arm 212. Shortly before or after this measurement (e.g., within about 1 hour or less while the person remains at rest), there may be a recordation of a blood flow waveform at the wrist 216 or other easily monitorable site of the person. Then, the blood flow waveform is continuously monitored and if specific changes in the blood flow waveform are noted (e.g., as predetermined or preset or satisfying or not satisfying certain thresholds), only then re-measure blood pressure via the pressure cuff 206 and restart this monitoring process.

In some embodiments, there may be battery-powered integrated device (e.g., see FIG. 2) that is configured to perform a cuff-based pressure measurement of systolic and diastolic blood pressure at the upper arm 214 or the wrist 216 and display systolic and diastolic measurements. This device can be configured to continuously (or frequently) monitor a blood flow in an artery of the person at that same site or at some other easily accessible site using a sensor (e.g., the blood flow sensor 106, a continuous-wave ultrasound Doppler blood flow sensor) mounted within a band attached to the patient over the artery to be sensed or having another wearable form factor worn on the person or contacting the person. The device can be configured to continuously analyze this blood flow waveform in real-time to determine whether there is a change in waveform shape which would indicate a significant change in blood pressure (or heart rate) and then instigate or request or instruct a further blood pressure (or heart rate) measurement via the pressure cuff 206 when a significant change has been detected, or when a considerable time has passed since the previous cuff-based measurement, as disclosed herein. Note that the pressure cuff 206 can be worn on the upper arm 214 or the wrist 216. Likewise, note that the blood flow sensor can be integrated within the pressure cuff 206, or the blood flow sensor may be mounted within a separate band (or another wearable form factor) placed at the wrist 216, the ankle, or the thigh of the person. Similarly, if the blood flow waveform in a forearm of the person is found to be affected significantly (e.g., as preset or predetermined in advance) by changing a flow demand to the arm 212 independently of any change in a calculated/estimated (virtual) or measured blood pressure, then a femoral artery of the person (e.g. at the thigh of the person) may be used to provide the blood flow waveform more reliably reflecting changes in a central blood pressure of the person.

In other embodiments, the simultaneous use of two or more blood flow sensors such as 106 or 210 is contemplated. The two or more blood flow sensors may be located at a single location (e.g., a sensor array), such as within a wearable as described herein. Alternatively, the two or more blood flow sensors may be located at different locations such as a head, a torso, a limb, the arm 212, the upper arm 214, the wrist 216, a finger, a toe, a thigh, an ankle, a leg or any part thereof. The output of each blood flow sensor may be individually used as described above to calculate/estimate a plurality of virtual blood pressure values. Alternatively, the output of each of the two or more blood flow sensors may be combined using a mathematical function or be a result of the mathematical function, such that the two or more sensors function as an equivalent single sensor which is used as described above to calculate/estimate a virtual blood pressure value. For example, the mathematical function can be a mean function, a median function, a mode function, a maximum function, a minimum function, a weighted function, or another mathematical function.

The processor 102 may determine a pressure change from a change in a flow waveform shape in various ways. For example, one of such ways can include deriving one or more parameters of a waveform shape (e.g., Doppler flow) for each heartbeat period and use the change in one or more parameters to indicate that the blood pressure may have changed. As shown in FIG. 4, some of these parameters can include a heartbeat period B, a height of an initial upswing H, a period of an initial upswing T, a frequency of an oscillatory component 1/C, or others.

In one mode of operation, there are certain assumptions including that (1) peripheral bed and proximal vessels do not change ‘tone’ and periphery maintains constant state of perfusion and (2) that vascular system is ‘linear’. As such, where the vascular system is linear (i.e. output responds proportionally to input) and periphery remains constant, detected flow should change proportionally to input pressure. This should provide the relationship that pressure and flow are related linearly so:
Foot to Peak flow proportional to minimum to maximum (pulse) pressure
Mean flow proportional to mean pressure
Initially Measure brachial pressure diastolic D(0) and systolic S(0)
   Measure foot F(0) and peak K(0) of flow waveform (e.g., Doppler flow waveforms)
   Measure average (mean) height of flow waveform (e.g., Doppler flow waveforms) M(0)

Derive Mean pressure Pm(0)=(S(0)+2D(0))/3

Pulse Pressure Pp(0)=(S(0)−D(0))

Then predict at a later time (t) the diastolic D(t) and systolic S(t) pressures from measurements F(t), K(t), and M(t) (e.g., Doppler measurements):

Pulse pressure: Pp(t)=Pp(0)×(K(t)−F(t))/(K(0)−F(0))

Mean pressure Pm(t)=Pm(0)×M(t)/M(0)

This can be rearranged to give:

S(t)=Pm(t)+Pp(t)×⅔

D(t)=Pm(t)−Pp(t)/3

Limitations:
Peripheral perfusion may vary irrespective of Blood pressure
Arterial system is not ‘linear’
Mean flow (e.g., Doppler flow) is not accurately determinable for low pulsatile flows

In one mode of operation, other ways to support the validity of these measurements or to replace them using other parameters of Doppler flow waveform or any waveform representing blood flow direct or indirectly. It has been observed that the blood flow (velocity) waveforms are usually strongly pulsatile when the subject is at rest (e.g., the wrist 216, the thigh, the ankle). The blood flow (e.g., velocity) waveform has an initial upswing following onset of cardiac systole which is the most observable characteristic of such waveforms. Further, this upswing has a height and time period which varies with blood pressure (but also possibly with changes in peripheral vascular tone). It is worth investigating the relationship between brachial blood pressure and resultant peripheral blood flow waveform shape to determine their likely relationships and their relative independence of peripheral vascular state.

Parameters suggested include:
Simple Height of the upswing
Time period of the upswing
Upswing gradient

Complex Waveform shape analysis
Waveform fitting to a simple electrical model driven by a basic driving function
Time dilation/compression (TDC)
Laplace modelling

However, it is clear that varying peripheral state with unchanging brachial blood pressure may also lead to such changes.

Modeling can be used as a method of identifying suitable parameters. Before practical investigation of possible parameters to indicate change in brachial pressure, it is useful to investigate an electrical model which incorporates the main known properties of the peripheral vascular system (see FIG. 5) and see if any particular parameter might be closely related to the change in driving blood pressure but robust to changes in peripheral state.

The simplest models for Driving Function Shape may be: Impulse or Square wave, defined by height and mark/space ratio.

The simplest electrical analogue is a lumped component circuit as shown in FIG. 6, though actual values of Rs, C, and L would differ from the example values shown:
Where Blood mass in vessel can be considered constant represented by inductance L
Vessel elasticity will vary with mean pressure represented by Capacitance C
Vessel resistance can be considered constant represented by Resistance
Vascular bed resistance will vary with peripheral state, again represented by Resistance

There is evidence that vessel compliance is inversely proportional to log(mean pressure), but we can assume for small changes that compliance is inversely proportional to mean pressure Pm. So Vessel compliance, equivalent to Capacitance=1/Pm, whilst inductance L is constant.

In this circuit, any oscillation created by an input pulse will have a frequency= 1/27 x sqrt(LC) which is notable for not being directly affected by the value of the resistances.

So, say frequency Fosc(0) is known at a mean pressure:

Pm(0)Fosc(0)=½π×sqrt(L/P(0))

Then at a later time ‘t’: Fosc(t)=½π×sqrt(L/(P(t))

Therefore: Pm(t)=Pm(0)×(Fosc(t)/Fosc(0))²

Accordingly, for example, a 5% change in frequency indicates a 10% change in mean pressure.

While the frequency of oscillation may be determined by a variety of complex procedures, it is postulated that a measure of the time from foot to peak of the flow waveform (T in FIG. 4) is a valid indicator of the oscillating frequency (Fosc proportional to 1/T) and a simplifying substitution can be made using initial flow upswing time T(0) and at a later time T(t).

Then: Pm(t)=Pm(0)×(T(0)/T(t))²

Using these assumptions, it is possible to predict mean blood pressure at a time (t) from an initial knowledge of brachial pressures and the change in frequency of oscillation or flow upswing time T of the blood flow waveform supplying the periphery.

This estimated change in mean blood pressure Pm(t)−Pm(0) may suffice as an indicator that the patient's blood pressure has changed and could be used alone as the indicator that a new pressure-cuff based measurement is required.

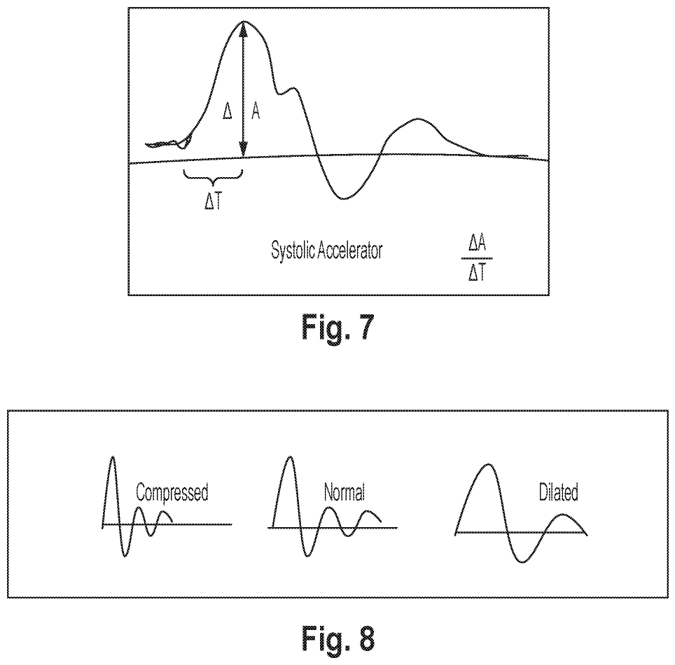

There may be more information in the flow waveform. The above technique only estimates changes in mean blood pressure, though the knowledge of Systolic and Diastolic pressures might be considered useful clinically. As we are using the assumption of linearity, then the height of the pressure waveform variation should be linearly related to the change in height of the flow waveform. So considering the systolic upswing from Pdias to Psys, this should be linearly related to the amplitude swing in flow measurements, Adias to Asys (and for Doppler signals, Vdias to Vsys, representative of velocity rather than amplitude) as shown in FIG. 7.

Following initial measurements at time 0, the constant of proportionality K can be derived:

$$\frac{Pdias ( 0 ) - Psys ( 0 )}{Adias ( 0 ) - Asys ( 0 )} = K$$

Further, we can use the common approximation of mean brachial blood pressure to relate the three pressure parameters.

Pmean=Psys/3+2×Pdias/3

These can be mathematically manipulated to give us Psys and Pdias at a future time (t) from those known at time (0):
Given that mean blood pressure at time t has already been estimated (above) by:

Pmean(t)=Pmean(0)×(Fosc(t)/Fosc(0))²
And K has already been derived, then:

Psys(t)=Pmean(t)+2×K(Adias(t)−Asys(t))/3

Pdias(t)=Pmean(t)−K(Adias(t)−Asys(t))/3

This would allow a continuous indication of both Systolic and Diastolic brachial pressures if required.

There may be further ways to estimate this frequency of oscillation. The shape of the waveform is akin to a damped sinusoid and may be estimated by various methods: Fourier analysis, Time Compression Ratio, or most simply by the time for the initial upswing to occur. As the simplest method of using foot to peak time of the flow waveform might be found sub optimal, two further methods are proposed:

### Curve Fitting—Time Compression/Dilation Analysis

Here it is proposed to take as reference the flow waveform for a complete cardiac cycle at time 0, and by curve fitting, stretch or compress in time the later flow waveform at time t to find a ‘best fit’ with the initial waveform. This could provide one single variable, Time Compression Ratio (“TCR”), which would encompass only the oscillatory components of the waveform and be unaffected by changes in non-pulsatile (mean) flow. Further, it does reflect the variation in waveforms frequently observed. For example, the three waveforms in FIG. 8 representing compressed, normal, and dilated signals.

This compression ratio (TCR=New timescale/Original timescale) can then be used in place of the foot-to-peak time ratio already considered above:

Replacing Pm(t)=Pm(0)×(T(0)/T(t))²

With Pm(t)−Pm(0)/TCR²

### Curve Fitting—Laplace Transform Modelling

Here a Laplace transform equation is used to describe the two flow waveforms based on an impulse driving function driving a second or third order Laplace transfer function (which mimics the production of a damped harmonic oscillation). The process is described in Skidmore R and Woodcock J P (1980) Ultrasound in Medicine & Biology V6 pp 7-10. Again, a ‘best fit’ approach is taken to obtain the appropriate parameters for both waveforms. The parameter affecting the frequency of oscillation (the imaginary part) can then be used in place of the Fosc ratio in estimating mean pressure change.

In an second embodiment, instead of deriving multiple parameters from the baseline blood pressure and baseline waveform associated with the baseline blood pressure, a linear relationship of one or more parameters of the blood flow waveforms can be determined from examining at least two waveforms taken at at least two different pressures. For example, the area under the waveform curve could be recorded for the current patients' BP and then for some number of incremental other (e.g., lower) pressures to set a rate of BP change based on that patient's physiology. By doing so this allows the system to minimize the physiologic variation of patients when determining their BP. Furthermore, the area under the curve would be averaged over several heartbeats as well. Other parameters, as disclosed elsewhere herein, could be used in conjunction with or in place of the area under the curve.

Once the system is calibrated (per this second method) then the patients BP could be followed digitally. Then, like the other embodiment, if there is a 5, 10, 20% change in the digital (or virtual) BP, then a standard mechanical BP might be performed. The threshold for check via a cuff BP would be seat by the end user. For retail products or outpatient monitoring (i.e. holder monitors/pacemakers) they would be linked via an app once to several times a day, again set by the end user.

Such systems might use a linear regression to determine mean arterial pressure based on the flow velocity of blood flow waveform, (e.g., a Doppler signal).

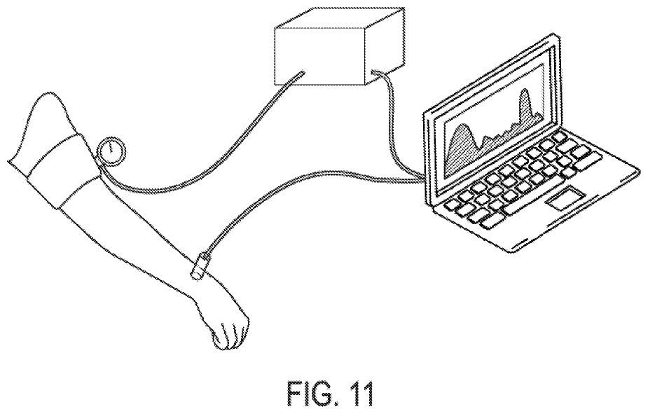

The second embodiment calibrates a pulse waveform either from Doppler, pulse volume, or any waveform that is in direct relationship to blood pressure. The waveform is correlated to, for example, the max amplitude of a patient's systolic BP and then the mean value under the curve to a patient's mean BP. By doing this we will be able with one digital signal follow patient's Heart Rate, Systolic, diastolic and mean MP's continuously without having to disturb patients with either an invasive arterial catheter placed invasively into a patients peripheral artery or the recurring uncomfortable BP cut. A generic setup of such a system is shown in both FIG. 2 and FIG. 11.

To implement the second embodiment, a BP cuff is inflated, all the while waveforms are being recorded. The waveform could be generated from such things as a Doppler, pulse volume recorder, or photo spectrometry, etc. The amplitude, area, (mean area) and HR (heart rate) are recorded and correlated with the final systolic BP—(cuff pressure) so that a graphic relationship is generated between a specific patient's [BP and augmented BP] to the simultaneous recorded waveform. This allows for the calculation of a slope of a line specific to that patient for that period of time. How often that slope is recalibrated is up to the end user, but may be set by such criteria as time (hourly, every 4, 6, 8, 12 hours), daily, or by changes in blood pressure (e.g., a change of 20 mmHg systolic, 10 mmHg mean, 20 mmHg mean, or even ranges e.g., SP<110 mmHG or SP>180 mmHg).

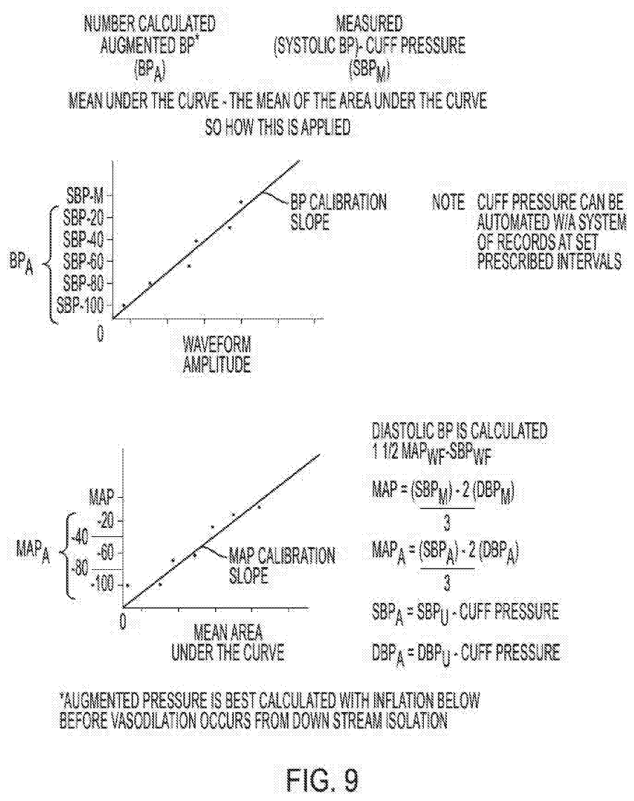

As the cuff inflates, amplitude, HR, and area under the curve for each heartbeat is recorded and stored. A user or manufacture may set the #of data points to collect. This is done until the patient's actual BP is reached knowing the waveform goes to zero. However, only two points are necessary to determine slopes of a patients waveform parameters. More points simply refine such measurements. Furthermore, this process does not necessarily have to follow an increase or a decrease in external cuff pressure, although there may be some benefits to preforming the process in one way versus another depending on the context of the measurement. The key is to obtain waveforms at at least two BPs. Further, while external pressure applied via a cuff is one way to ensure that waveforms are captured at two or more BP points, it is not the only way. An app or other software driven vehicle may be used to instruct a patient to perform two or more activities ensuring that the resultant BP between the activities would presumably be different. At the completion of the activities or during said activities, waveform are captured and while the actual BP would not be known, the linear relationship of a patient's BP would be calculated. FIG. 9 illustrates such a set of measurements. As shown in FIG. 9, the augmented pressure BPA is best calculated with inflation before vasodilation occurs from downstream isolation. To gather data points the cuff pressure can be automated with a system of records at set prescribed intervals. The measured (systolic BP)—Cuff pressure is represented as SBP_{M}. The mean under the curve minus the mean of the area under the curve. The diastolic BP is calculated as 1½ MAP_{WF}−SBP_{WF}. The relationship of MAP, MAP_{A}, SBP_{A}, and DBP_{A} is provided by:

$$MAP = \frac{( {SBP}_{M} )}{3} - 2 ( {DBP}_{M} )$$

$${MAP}_{A} = \frac{( {SBP}_{A} )}{3} - 2 ( {DBP}_{A} )$$

$${SBP}_{A} = {SBP}_{U} - CUFF PRESSURE$$

$${DBP}_{A} = {DBP}_{U} - CUFF PRESSURE$$

Other embodiments consider the pulsicity of the blood flow waveform. Pulse generation is based on the principle that when the ventricle contracts and creates a needed pressure gradient, a volume of blood is rapidly ejected into the arterial vessel. The aorta and arteries have a lower resistance to blood flow when compared to the arterioles and capillaries. The elastic recoil of the arteries forces the blood out into the arterioles during the diastole.

The resistance index measures the pulsatile blood flow that reflects the resistance to blood flow caused by microvascular bed distal to the site of measurement. The RI is typically measured by Doppler sonography in an intrarenal artery and is calculated according to:

$$RI = \frac{{v}_{systole} - {v}_{diastole}}{{v}_{systole}}$$

The normal RI for an adult is about 0.7. An RI of 1 indicates systolic flow with no diastolic flow; and an RI greater than 1 indicates reversed diastolic flow.

A pulsatility index (PI) velocity, also known as the Gosling index, can be generated from the waveform. PI is calculated from a pulsed Doppler waveform according to Equation 3 below.

$$PI = \frac{{v}_{max} - {v}_{min}}{{v}_{mean}}$$

The PI is also known as the reverse/forward flow index, and demonstrates the pulse amplification (Vmax−Vmin) and arterial stiffness. The systolic blood pressure is approximately equal to the pulse amplification minus the pulse amplitude.

In the methods and systems of this disclosure, MAP may be determined based on the AUC, or “work.” Work is the force over a distance, and force is mass times acceleration. In an exemplary embodiment, the force curve is a Doppler waveform; and the mass is blood that has both forward movement and reverse movement. Blood pressure is the force represented by the slope for the waveform per moment in time. The amplitude represents the mass or volume of blood; and the distance is the visual spectrum of the probe. The work is the summation of the AUC of the waveform.

In exemplary embodiments, the baseline mean arterial pressure is calculated with a modified Bernoulli equation, where P is the pressure, V₁ is the pre-orifice velocity, and V₂ is the post-orifice velocity.

The Bernoulli equation is a conservation of energy principle that is appropriate for flowing fluids, such as blood. The term “Bernoulli effect” is the lowering of fluid pressure regions where the flow velocity is increased. In exemplary embodiments, the pressure is calculated from the Bernoulli equation when the diameter of the pipe or vessel is known.

FIG. 9, but with (Vₘₐₓ−Vₘᵢₙ) amplitude along the horizontal axis instead of “waveform” amplitude, demonstrates a linear regression of systolic blood pressure (SBP) to the pulse amplification (Vₘₐₓ−Vₘᵢₙ). In this embodiment, the patient fitted blood pressure is the slope of the linear regression. As stated above, the Bernoulli equation assumes patient fitted blood pressure should remain constant. Accordingly, the systems and methods according to the disclosure detect for changes to the slope. Using the pulsatiling index may offer more advantages in critically ill patients. V_{elease f}−pulsatiling Index velocity or (Vₘₐₓ−Vₘᵢₙ)/Vₘₑₐₙ and systolic BP ˜(Vₘₐₓ−Vₘᵢₙ) −pulsatiling amplitude.

In this embodiment, the MAP would be relative to (Vₘₐₓ−Vₘᵢₙ)/Vₘₑₐₙ instead of the mean area under the curve. And the associated calculations for FIG. 9, would be as follows:

$$Diastoic BP is calculated 1 \frac{1}{2} {MAP}_{\text{?}} - {SBP}_{\text{?}}$$

$${MAP}_{\text{?}} = \frac{( {SBP}_{\text{?}} )}{3} - 2 ( {DBP}_{\text{?}} )$$

$${MAP}_{\text{?}} = \frac{( {SBP}_{\text{?}} )}{3} - 2 ( {DBP}_{\text{?}} )$$

$${SBP}_{\text{?}} = {SBP}_{\text{?}} - CUFF PRESSURE$$

$${DBP}_{\text{?}} = {DBP}_{\text{?}} - CUFF PRESSURE$$

$$\text{?} \text{indicates text missing or illegible when filed}$$

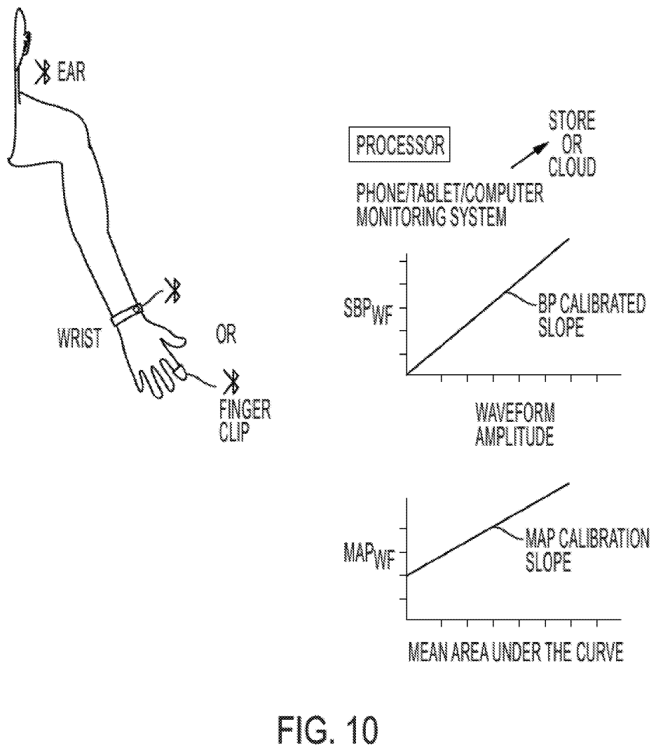

Once a linear relationship is established for a patient (i.e., a patient specific relationship) continuous blood pressure monitoring can take place. This could be done for personal use by utilizing for example wrist bands, finger clips, ear clips that interface via Bluetooth to smart device with a processor (phone tablets/computer) in which the data could be stored locally or on the cloud. See FIG. 10. For ambulatory medical application to follow patient's BP while going through their daily activity linking this to such things as halter monitor, pace maker, cardiac monitors, cardiac electric stimulators, ICDs via finger clip, ear clip, or wrist bands with for example Bluetooth. And for Medical/Critical care to have a continuous digital vital sign monitor via wrist bands, finger clip, ear clip, etc, that interfaces by Bluetooth (or other communication protocol) to a monitor system that is linked to a patient's medical record.

In a third embodiment, no baseline or calibrating BP measurements is made. That is, the is no externally applied pressure or application to instruct the user to perform activities, so to artificially apply or raise/lower a user's BP. Instead, an assumed general BP waveform mapping is used to allow for a calculation of a virtual BP. The more data from a large set of user's will help to establish a more robust map. In use, the actual or measured BP may never be known, although it may be easily calculated from the monitored waveforms. Instead the method of the third embodiment, relies on monitoring parameter changes of the waveforms based on the general BP waveform mapping.

Various techniques for measuring blood pressure may be used to forecast, diagnose, monitor, or treat a medical condition, disorder, or disease of a human, an animal, a pet, a fish, or a bird, whether unborn, born, baby, infant, toddler, preschooler, kid, teen, adult, elderly, paralyzed, wheel-chair bound, or others. For example, some medical conditions, disorders, or diseases can include arthritis, hypo and hypertension, asthma, cancer (e.g. lung, bronchus, prostate, colon, rectum, breast, liver, pancreas, gallbladder, bladder, endometrial, kidney, leukemia, melanoma, non-Hodgkin lymphoma, thyroid, or others), diabetes (e.g. type 1 and type 2), bronchitis, coronary heart disease, dementia, Parkinson's disease, Alzheimer's disease, epilepsy, multiple sclerosis, osteoporosis, stroke, heart attack, chronic kidney disease, deep vein thrombosis, shingles, acne, anxiety, sleep apnea, atherosclerosis, nephritis, nephrotic syndrome, skin burn, nephrosis, gallstones, jaundice, cirrhosis, dyspepsia, gastric ulcer, infectious disease (e.g. flu or others), scoliosis, spondylosis, spinal stenosis, bone fracture, ischemic heart disease, or others.

It is to be understood that the above description is intended to be illustrative, and not restrictive. For example, the above described embodiments (and/or aspects thereof) may be used in combination with each other. In addition, many modifications may be made to adapt a particular situation or material to the teachings of the subject matter without departing from its scope. While the dimensions and types of materials described herein are intended to define the parameters of the subject matter, they are by no means limiting and are exemplary embodiments. Many other embodiments will be apparent to one of ordinary skill in the art upon reviewing the above description. The scope of the subject matter described herein should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112, sixth paragraph, unless and until such claim limitations expressly use the phrase “means for” followed by a statement of function void of further structure.

This written description uses examples to disclose several embodiments of the subject matter, including the best mode, and also to enable any person of ordinary skill in the art to practice the embodiments disclosed herein, including making and using any devices or systems and performing any incorporated methods. The patentable scope of the subject matter is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

The foregoing description of certain embodiments of the disclosed subject matter will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (for example, processors or memories) may be implemented in a single piece of hardware (for example, a general purpose signal processor, microcontroller, random access memory, hard disk, and the like). Similarly, the programs may be stand-alone programs, may be incorporated as subroutines in an operating system, may be functions in an installed software package, and the like. The various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present subject matter are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

Since certain changes may be made in the above-described systems and methods, without departing from the spirit and scope of the subject matter herein involved, it is intended that all of the subject matter of the above description or shown in the accompanying drawings shall be interpreted merely as examples illustrating the concepts herein and shall not be construed as limiting the disclosed subject matter.

Various embodiments of the present disclosure may be implemented in a data processing system suitable for storing and/or executing program code that includes at least one processor coupled directly or indirectly to memory elements through a system bus. The memory elements include, for instance, local memory employed during actual execution of the program code, bulk storage, and cache memory which provide temporary storage of at least some program code in order to reduce the number of times code must be retrieved from bulk storage during execution.

I/O devices (including, but not limited to, keyboards, displays, pointing devices, DASD, tape, CDs, DVDs, thumb drives and other memory media, etc.) can be coupled to the system either directly or through intervening I/O controllers. Network adapters may also be coupled to the system to enable the data processing system to become coupled to other data processing systems or remote printers or storage devices through intervening private or public networks. Modems, cable modems, and Ethernet cards are just a few of the available types of network adapters.

The present disclosure may be embodied in a system, a method, and/or a computer program product. The computer program product may include a computer readable storage medium (or media) having computer readable program instructions thereon for causing a processor to carry out aspects of the present disclosure. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing..

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, among others. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions. The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Words such as “then,” “next,” etc. are not intended to limit the order of the steps; these words are simply used to guide the reader through the description of the methods. Although process flow diagrams may describe the operations as a sequential process, many of the operations can be performed in parallel or concurrently. In addition, the order of the operations may be re-arranged. A process may correspond to a method, a function, a procedure, a subroutine, a subprogram, etc. When a process corresponds to a function, its termination may correspond to a return of the function to the calling function or the main function.

Features or functionality described with respect to certain example embodiments may be combined and sub-combined in and/or with various other example embodiments. Also, different aspects and/or elements of example embodiments, as disclosed herein, may be combined and sub-combined in a similar manner as well. Further, some example embodiments, whether individually and/or collectively, may be components of a larger system, wherein other procedures may take precedence over and/or otherwise modify their application. Additionally, a number of steps may be required before, after, and/or concurrently with example embodiments, as disclosed herein. Note that any and/or all methods and/or processes, at least as disclosed herein, can be at least partially performed via at least one entity or actor in any manner.

Although preferred embodiments have been depicted and described in detail herein, skilled artisans know that various modifications, additions, substitutions and the like can be made without departing from spirit of this disclosure. As such, these are considered to be within the scope of the disclosure, as defined in the following claims.

What is claimed is:

1. A method comprising:
instructing, by a processor, a sensor worn on a target to monitor a first blood flow of the target during a time period including a cardiac cycle of the target such that the sensor generates a first set of waveform data representing the cardiac cycle during the time period, wherein the first set of waveform data is associated with a blood pressure measurement;
receiving, by the processor, a second set of waveform data for a second blood flow from the sensor after the first set of waveform data is generated by the sensor;
identifying, by the processor, a parameter change in the second set of waveform data relative to the first set of waveform data;
correlating, by the processor, the parameter change to the blood pressure measurement such that a virtual blood pressure for the second blood flow is generated; and
taking, by the processor, an action based on the virtual blood pressure.

2. The method of claim 1, wherein the blood pressure measurement is a first blood pressure measurement, wherein the action includes instructing a blood pressure monitor to obtain a second blood pressure measurement that is different from the first blood pressure measurement.

3. The method of claim 2, wherein the processor instructs the blood pressure monitor to obtain the first blood pressure measurement.

4. The method of claim 1, wherein the parameter change is a change in a waveform shape.

5. The method of claim 1, wherein the sensor is a continuous-wave sensor.

6. The method of claim 5, wherein the target has a finger, a toe, a wrist, an ankle, or a thigh, wherein the continuous-wave sensor is worn on at least one of the finger, the toe, the wrist, the ankle, or the thigh of the target.

7. The method of claim 5, wherein the target wears a pressure cuff enabling the blood pressure measurement, wherein the pressure cuff includes the sensor.

8. The method of claim 1, wherein the processor identifies the parameter change based on deriving a first parameter of a first waveform shape for each cardiac cycle during the first blood flow, deriving a second parameter of a second waveform shape for each cardiac cycle during the second blood flow, and identifying a change between the first parameter and the second parameter.

9. The method of claim 8, wherein each of the first parameter and the second parameter is a heartbeat period.

10. The method of claim 8, wherein each of the first parameter and the second parameter is a height of an initial upswing.

11. The method of claim 8, wherein each of the first parameter and the second parameter is a period of an initial upswing.

12. The method of claim 8, wherein each of the first parameter and the second parameter is a frequency of an oscillatory component.

13. A device comprising:
a processor programmed to:
instruct a sensor worn on a target to monitor a first blood flow of the target during a time period including a cardiac cycle of the target such that the sensor generates a first set of waveform data representing the cardiac cycle during the time period, wherein the first set of waveform data is associated with a blood pressure measurement;
receive a second set of waveform data for a second blood flow from the sensor after the first set of waveform data is generated by the sensor;
identify a parameter change in the second set of waveform data relative to the first set of waveform data;
correlate the parameter change to the blood pressure measurement such that a virtual blood pressure for the second blood flow is generated; and
take an action based on the virtual blood pressure.

14. The device of claim 13, wherein the blood pressure measurement is a first blood pressure measurement, wherein the action includes instructing a blood pressure monitor to obtain a second blood pressure measurement that is different from the first blood pressure measurement.

15. The device of claim 13, wherein the parameter change is a change in a waveform shape.

16. The device of claim 13, wherein the sensor is a continuous-wave sensor.

17. The device of claim 16, wherein the target wears a pressure cuff enabling the blood pressure measurement, wherein the pressure cuff includes the sensor.

18. The device of claim 13, wherein the processor identifies the parameter change based on deriving a first parameter of a first waveform shape for each cardiac cycle during the first blood flow, deriving a second parameter of a second waveform shape for each cardiac cycle during the second blood flow, and identifying a change between the first parameter and the second parameter.

19. The device of claim 18, wherein each of the first parameter and the second parameter is a heartbeat period.

20. The device of claim 18, wherein each of the first parameter and the second parameter is a height of an initial upswing.

21. The device of claim 18, wherein each of the first parameter and the second parameter is a period of an initial upswing.

22. The device of claim 18, wherein each of the first parameter and the second parameter is a frequency of an oscillatory component.

23. The method of claim 1, wherein the action includes instructing an output device or a transmitter to respectively output or transmit the virtual blood pressure or a virtual heart rate.

24. The device of claim 13, wherein the action includes instructing an output device or a transmitter to respectively output or transmit the virtual blood pressure or a virtual heart rate.
